(19) United States
(12) Patent Application Publication
(10) Pub. No.: US 2020/0188055 A1
(43) Pub. Date: Jun. 18, 2020

(54) Surgical Safety Devices and Methods

(71) Applicant: James Keith Bleiler, East Falmouth, MA (US)

(72) Inventor: James Keith Bleiler, East Falmouth, MA (US)

(21) Appl. No.: 15/721,938

(22) Filed: Oct. 1, 2017

(51) Int. Cl. A61B 90/00

(52) U.S. Cl. CPC A61B 90/08; A61B 2017/00022; A61B 90/03

(57) ABSTRACT

A surgical safety device for use in connection with a powered surgical instrument has a controller in electrical communication with a powered surgical instrument. The controller is connected to sensors that can provide information to allow the controller to determine that a patient's limb is about to move or has started to move unexpectedly. Upon making this determination, the controller can shut off the powered surgical instrument so that the patient is not injured.

### CROSS-REFERENCE TO RELATED APPLICATIONS

None.

### FIELD OF INVENTION

The present disclosure relates to a surgical safety device that utilizes electromyography (“EMG”) sensors to detect patient movement during surgery. More specifically, the present disclosure is directed to surgical safety devices and methods that are particularly adapted for use with cautery instruments during transurethral resection procedures.

### BACKGROUND OF INVENTION

Modern surgical equipment and techniques often require a surgeon to perform surgical operations or procedures on patients that can experience sudden movements on an operating table. Sudden movements may be voluntary or induced and represent a potential risk from the surgical instruments that are utilized in such operations. Modern surgical instruments include lasers, drills, electrocautery, harmonic scalpel, argon beam coagulator, or other similar instruments. During applied power, the action of these instruments may also, in and of themselves, induce patient or tissue movement via nerve or muscle stimulation. These instruments can cause irreversible injury when they misaligned, misapplied, or inappropriately moved by such voluntary or involuntary movement.

The risk of injury is particularly acute in surgical procedures that involve the use of an electrosurgical resectoscope. In the diagnosis and initial treatment of bladder cancer, for example, surgical procedures such as transurethral resection (TUR) are common. The aim of TUR is to remove all visible lesions and part of the underlying muscle tissue. Unfortunately, the obturator nerve passes close to the bladder wall and, if stimulated by the electrical current transmitted by the resectoscope during the removal of any lesions, can cause the patient's leg to jerk involuntarily, quickly, and unexpectedly. This is commonly referred to as obturator reflex, and it creates a substantial risk of bladder perforation, deep bladder wall tissue injury, nearby organ injury, or some other unintended injury to the patient, such acute vascular injury of the pelvic vessels, which can be life-threatening. Power to the cutting electrode is routinely voluntarily activated by depressing a foot pedal or depressing a hand switch. During activation, this reflex can occur during use of the powered instrument. It is nearly impossible to humanly detect this movement and voluntarily and emergently cease power to the cutting electrode before the sudden violent leg movement drives the cutting electrode deeper into the tissue being cut.

Traditionally, surgeons have used various techniques, including nerve blocks and induced paralysis, to address such problems. However, these techniques increase time and cost and have additional potential complications. Moreover, they are usually implemented only after a reflexive incident and unintended tissue damage has occurred.

Once a surgeon orders nerve blocks or induces paralysis, an anesthesiologist will often have to engage in more intensive airway management (e.g., intubation rather than laryngeal mask ventilation) as well as more intensive management of blood pressure. Medications to induce paralysis have a comprehensive list of potential complications that are well known but essentially will result in, at best, a longer recovery time for the patient and, in some cases, additional complications.

The risks associated with sudden movements by patients in close proximity to dangerous surgical instruments create major hazards in the operation room. Currently, there is no simple safety device available to minimize these risks and/or to prevent these unnecessary injuries. What is needed, then, is a medical surgery safety device that addresses these problems.

### SUMMARY OF INVENTION

This summary is provided to comply with 37 C.F.R. § 1.73, requiring a summary of the invention briefly indicating the nature and substance of the invention. It is submitted with the understanding that it will not be used to interpret or limit the scope or meaning of the claims.

The present disclosure is directed to a medical surgery safety device used in connection with a surgical instrument. The medical surgery safety device includes an electromyography skin surface electrode to detect electrical activity in muscle in response to voluntary or induced stimulation of the nerve causing limb movement. A controller receives information from the electromyography sensor and controls the surgical instrument. In certain embodiments, the controller deactivates the power to the surgical instrument after communicating with the electromyography sensor and detecting electrical activity in response to stimulation of the leg muscle by the nerve.

Additional features found in the present disclosure include a medical surgery safety device that includes an accelerometer for attachment to a limb to detect movement of the limb in response to stimulation of the muscle group moving that limb by the nerve with the controller receiving a signal from the accelerometer. The medical surgery safety device can include a housing for holding the controller and a reset button assembly connecting to the controller and extending through the housing with the reset button having the ability to reset the controller.

The medical surgery safety device can include one or more electromyography sensors for attaching to a patient's limbs connected to the controller or one or more accelerometers for attaching to a patient's limbs connected to the controller.

The medical surgery safety device can include a display device for displaying information relating to the electromyography sensor. The controller is a microcontroller. The controller can connect to a plurality of single channel EMG boards with each EMG board transmitting a single channel of EMG sensor data that corresponds to the movement of a patient to the controller.

The instrument can be a cautery handpiece. The cautery handpiece can connect to a cautery console for monitoring the cautery handpiece, which can be connected to the controller which has the ability to shut off the cautery console and the cautery handpiece. A relay or other disconnect device can connect the cautery handpiece to the cautery console, so that the controller can deactivate the cautery console and the cautery handpiece.

The medical surgery safety device can include a USB Interface for receiving information from the controller. The medical surgery safety device can connect to and be powered by a power supply selected from the group consisting of any reasonable medical grade power supply including, but not limited to, AC or DC power, a battery pack or a USB interface.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 is a diagram of an over-head view illustrating the placement and arrangement of a medical surgery safety device in an operating room environment.

FIG. 2 is a block diagram of an embodiment of a surgical system.

FIG. 3 is a top plan view of an embodiment of a surgical safety device.

FIG. 4 is a cut-away plan view of the top half of the surgical safety device shown in FIG. 3 that has been opened to reveal the contents of the top half of the interior of the device enclosure.

FIG. 5 is a cut-away plan view of the bottom half of the surgical safety device shown in FIG. 3 that has been opened to reveal the contents of the bottom half of the interior of the device enclosure.

### DETAILED DESCRIPTION OF THE INVENTION

This disclosure is directed to example systems and methods for implementing a surgical safety device that uses EMG sensors to detect nerve stimulations that may cause muscle movements during surgical operations. In the following description, for purposes of explanation, numerous specific details are set forth in order to provide a thorough understanding of example embodiments. It will be evident to one skilled in the art, however, that embodiments can be practiced without these specific details. The description is not to be taken in a limiting sense, but is made merely for the purpose of illustrating the general principles of the invention, since the scope of the invention is best defined by the claims included herein.

In certain embodiments, surgical safety devices constructed in a manner consistent with the present disclosure use EMG detection and, optionally, accelerometers to detect unexpected bodily movements in patients during surgical procedures. Upon detection of any such movements, the disclosed safety device and method will shut off power to certain electrical surgical instruments to protect the patient from accidental harm.

In embodiments, the disclosed surgical safety device uses EMG detection sensors to measure muscle response or electrical activity in a muscle in response to nerve stimulation. EMG detection sensors include one or more small needles or electrodes that can be inserted into the skin only, through the skin into the muscle, or can take the form of a disposable gelled ECG/EMG surface sticker monitoring electrode to detect electrical activity. The sensors can use the signal detected by any of these sensors to send signals to controllers, computing systems, computing devices, and/or display devices for analysis. In certain embodiments, such devices can be used to shut off, override, or otherwise manipulate surgical devices to prevent them from injuring patients when such movements occur.

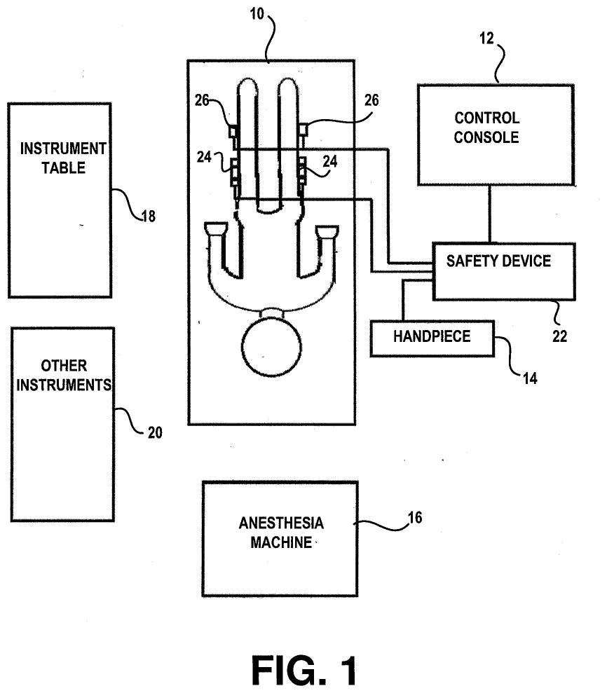

FIG. 1 depicts a typical medical surgery operating room (OR) environment. The medical equipment in the OR often includes an operating table 10, a control console 12, a powered tool handpiece 14, an anesthesiology machine 16, an instrument table 18, and other surgical instruments 20, such as a laser or endoscope for example.

In a typical procedure, such as a TUR, a surgeon uses a powered tool handpiece 14 and various other instruments to perform the procedure. An anesthesiologist operates an anesthesia machine 16 to keep the patient appropriately sedated. A nurse and a scrub technician can work near the powered tool control console 12 and the instrument table 18 to assist the surgeon. The OR can include other medical professionals and medical equipment, depending on the particular type of surgical procedure.

In certain embodiments of the present invention, a surgical safety device 22 is placed between the powered tool control console 12 and the handpiece 14, such as a cauterizing handpiece. The surgical safety device 22 functions as a protective interface that both monitors for motion or impending motion of a patient's limb and controls the power supply to the handpiece 14. In the event of any unexpected firing of a muscle, the surgical safety device 22 deactivates or cuts the power supply to the handpiece 14 and, in some embodiments, the control console 12, to prevent injury to the patient.

In certain embodiments, the surgical safety device 22 monitors sensors that are providing information about the patient, including one or more EMG sensors 24 and, optionally, one or more accelerometers 26. In this exemplary embodiment, the EMG sensors are attached to the patient's adductor muscles to detect electrical activity in the muscle indicating that the patient is about to move in some way.

In certain embodiments, the accelerometers 26 are attached to the patient's thighs. In other embodiments, the accelerometers are attached as close to the knees as possible. The accelerometers 26 detect movement of the legs or other monitored body parts, such as might occur from triggering the obturator nerve and causing a reflex action to occur.

The detection of the electrical activity in the muscle or sudden unexpected movement will trigger the surgical safety device 22 to shut off the cauterizing handpiece 14. In this exemplary embodiment, power flows from the control console 12 to the handpiece 14 through the surgical safety device 22. When the surgical safety device 22 detects indicators, signals, readings, and/or other information that indicate a rapid change in muscle electrical potential or leg velocity, the surgical safety device 22 interrupts the flow of power from the control console 12 to the handpiece 14.

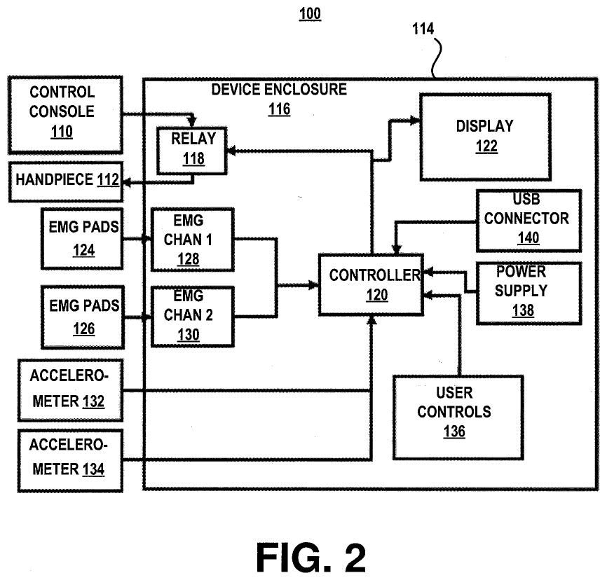

FIG. 2 depicts a block diagram of certain embodiments of a surgical system 100 that can implement features of the disclosed invention. In certain embodiments, the surgical system 100 is configured to perform cauterization, which can be used during bladder surgery, prostate surgery, transurethral resection procedures, and many other surgical procedures.

In embodiments, the surgical system 100 can be a cauterization system that includes a control console 110, a handpiece 112, such as a cautery instrument, and a surgical safety device 114 sitting electrically in between the control console 110 and the handpiece 112. The surgical safety device 114 functions as a protective interface that, upon receiving information about unexpected motion or potential motion of a patient's limb, quickly deactivates the handpiece 112 to prevent unintended cutting of tissue. In certain embodiments, the surgical safety device 114 cuts power to the tool 112 when it detects unexpected motion or possible motion of a patient. In certain embodiments, the device 114 cuts power to the tool 112 within a fraction of a second. In further embodiments, the device 114 cuts power to the tool 112 within 0.2 seconds. In this way, the surgical safety device 114 functions as a safety interface between the control console 110 and the powered tool 112. In other embodiments, the surgical safety device can instead be placed between a power source, such as an AC outlet and the control console 110, whereby power can be disrupted to the surgical system as a whole instead of just to the handpiece.

In certain embodiments, the surgical safety device 114 includes a housing or device enclosure 116 that contains a relay 118 positioned between the control console 110 and the handpiece 112 that can allow or block electricity and/or information in response to certain conditions. In embodiments, the device enclosure 116 contains a controller 120 and a display device 122 therein. In this embodiment, the controller 120 can be a programmable microcontroller that can process multiple channels of signals.

The display 122 on the device enclosure 116 can include any type and/or form of suitable hardware, software, or combination of hardware and software to support, enable or provide indications that the surgical system 100, the control console 110, the handpiece 112, and/or the surgical safety device 114 must be reset or is in need of power. In this embodiment, the display 122 can include one or more indicators that indicate power status and/or reset status for the surgical safety device 114.

In certain embodiments, the surgical safety device 114 is in electrical communication with one or more EMG pads 124, 126 that are placed on a limb where undesired movement could occur. For example, a tumor to be resected on the right side of the bladder could potentially case right-sided obturator reflex and thus the right leg would be monitored for movement or muscle recruitment. The one or more EMG pads 124, 126 enclose EMG sensors that connect to the controller 120 in the device enclosure 116 through one or more EMG boards 128, 130. In certain embodiments, the EMG board 128 is a single channel EMG sensor processing device that can capture EMG sensor data that corresponds to the movement of a patient. In other embodiments, there can be multiple channel EMG boards without deviating from the invention.

In embodiments, the controller 120 can monitor one or more EMG pads 124, 126 and one or more EMG boards 128, 130 to detect actual or imminent leg movements. The EMG sensors within the EMG pads 124, 126 can be attached to a patient's legs to allow the controller 120 to monitor the adductor muscles therein. The controller 120 can use the sensors to detect electrical activity in the muscle indicating that a patient is about to move, even when the patient is unconscious. In certain embodiments, the controller 120 receives signals obtained by direct sensing of a muscle and/or nerve group by the monitoring electrodes in the EMG pad or pads 124, 126. In certain embodiments, the EMG pads are comprised of cutaneous gelled monitoring electrodes, although other types of sensors can be used without deviating from the invention.

In certain embodiments, the EMG pads 124, 126 would record background electrical activity within a muscle. In the event of a spike in activity, which may indicate imminent movement of the patient's limb, the controller 120 cuts power to the handpiece 112 to avoid unintended injury. In further embodiments, detection of activity in the range of five to ten percent above a baseline of activity will cause the controller to cut power. In still further embodiments, detection of electrical activity in the range of seven percent above a baseline of activity will trigger a cut in power to the handpiece 112.

Optionally, the surgical safety device 114 can be connected to one or more accelerometers 132, 134. In embodiments, these accelerometers 132, 134 can be positioned on a patient's leg on or adjacent to the kneecap to detect movement of the leg. Alternatively, the accelerometers can detect changes in velocity and acceleration of the limb to which they are attached. If motion is detected, the one or more accelerometers send signals that include information relating to the movement to the controller 120 within the device enclosure 116.

In embodiments, the controller 120, in turn, monitors the accelerometers 132, 134 to detect unexpected limb movements in patients. The controller 120 can use the information either separately, or in conjunction with, the sensors in the EMG pads 124, 126 to detect or predict impending or actual limb movement. Upon detection of such sensor readings, the controller 120 can interrupt power to the handpiece 112 through the use of a relay or other similar mechanism as known to those of skill in the art.

The controller 120 can be implemented by software, hardware, firmware or a combination thereof. For example, the controller 120 can include components implemented by computer-executable instructions that are stored on one or more computer-readable storage media and that are executed to perform various steps, methods, and/or functionality in accordance with aspects of the described subject matter.

The controller 120 can store sensor readings in memory to analyze the sensor readings from the one or more accelerometers 132, 134 and/or the sensors in the one or more EMG pads 124, 126 for indications or precursors of patient movement. The controller 120 can implement one or more algorithms to detect, identify, or predict patient movements. In some embodiments, the algorithm or algorithms can account for factors, such as sensor placement variation. Other algorithms may allow certain “slow” intentional movement such as surgeon repositioning patient and only disconnect power upon the occurrence of “fast” or sudden movement. In still other embodiments, the controller 120 can communicate with the display device 122 to enable the display of output relating to the accelerometers 132, 134 and/or the sensors in the EMG pads 124, 126.

In embodiments, the EMG sensors in the EMG pads 124, 126 can be sensitive to variation in electrode placement, so that the controller 120 can store and compare sensor readings to an average of the samples over a predetermined time period, such as five seconds, to account for such variations. The controller 120 can be set to activate a protective circuit, in embodiments, this may actuate a relay 118 when sensor readings exceed the windowed average by a predefined threshold, such as seven percent, by way of example.

In embodiments, the controller 120 can be connected to user controls 136 that are housed within or on the device enclosure 116. The user controls 136 can include one or more buttons for powering up the surgical safety device 114 and one or more buttons for resetting the surgical safety device 114. In this exemplary embodiment, the controller 120 must wait for a reset signal from the user controls 136 before the relay 118 can reconnect the control console 110 to the handpiece 112. In embodiments, there may also be user controls 136 for changing the sensitivity of the controller such that it may cut power upon detection of either more or less electrical impulse activity. Where accelerometers are used, the user controls 136 may offer the user the ability to make the controller 120 more or less sensitive to movement of a limb detected by an accelerometer.

The controller 120 can be powered by a power supply 138 or by a USB connector 140. The power supply 138 can be an internal power source or an external power source. The USB connector 140 can connect to an external computer system or computing device (not shown).

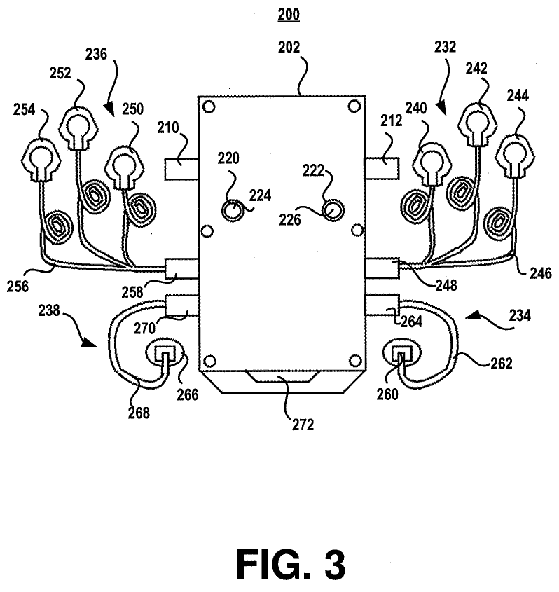

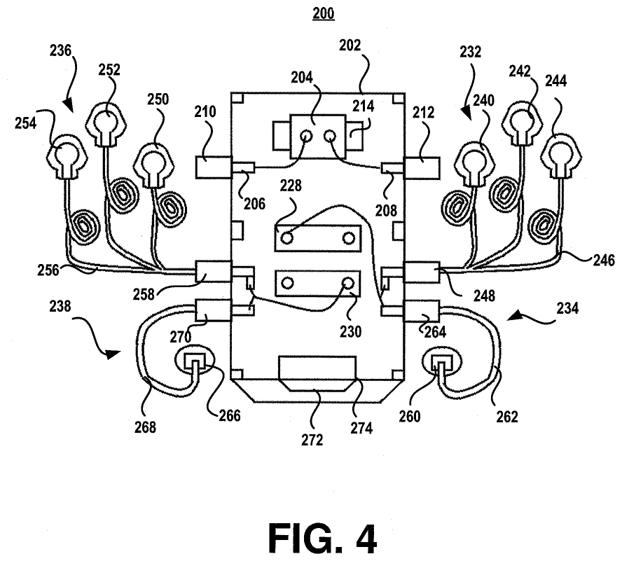

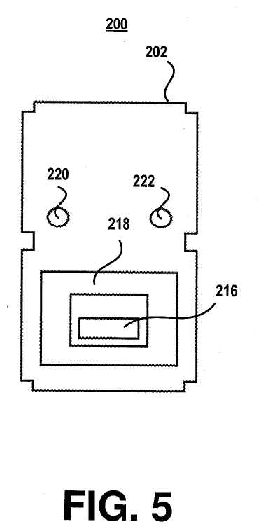

FIGS. 3-5 depict an embodiment of a surgical safety device 200 for controlling surgical instruments in a safe manner. The surgical safety device 200 can implement features of the surgical safety device 22 shown in FIG. 1 and/or the surgical safety device 114 shown in FIG. 2. In this embodiment, the surgical safety device 200 is particularly adapted for use with a cauterization system, such as the cauterization system 100 shown in FIG. 2, but is suitable for other surgical systems that utilize cautery instruments or other powered cutting instruments.

The surgical safety device 200 can function as a protective interface that uses EMG measurements and other related measurements to detect induced or voluntary nerve excitations that can cause sudden muscle movements in conscious or unconscious patients during surgery. Upon detection of such nerve excitations, the surgical safety device can override surgical instrument systems and deactivate cutting instruments, such as by deactivating power to the handpiece 14 shown in FIG. 1 or the handpiece 112 shown in FIG. 2. In this exemplary embodiment, the surgical safety device 200 detects impending motion of the limb of an unconscious patient and can quickly deactivate the handpiece 14 or the handpiece 112 to prevent injury.

In certain embodiments, the surgical safety device 200 includes a housing 202 that holds a relay and control board 204 that can allow electricity and/or information to flow from a control console 110, such as cautery console shown in FIG. 1, to a surgical instrument, such as the handpiece 112 shown in FIG. 1. The housing 202 can be an essentially rectangular device enclosure formed from metal, plastic, ceramic, or composite materials that are formed from conventional manufacturing methods or techniques.

In certain embodiments, the relay and control board 204 can be connected to a pair of terminals 206, 208 that include banana plugs 210, 212. The banana plugs 210, 212 can be connected to the control console 110 and the handpiece 112 to facilitate the flow of power from the control console 110 to the handpiece 112. In some embodiments, the control console 110 can receive or exchange information with the handpiece 112, so that the control console 110 can control the handpiece 112 at the direction of a surgeon or a surgical technician. The relay and control board 204 are positioned on a mount 214. Those of skill in the art will recognize that use of the disclosed invention with other powered tools could require a different electrical setup, such as additional relays for example.

The relay and control board 204 can be controlled by a microcontroller 216 that is positioned within the housing 202, so that the relay can be used to shut off the power to the handpiece 112 when necessary. The microcontroller 216 can be powered by an internal battery pack 218 within the housing 202 to allow the surgical safety device 200 to function as a standalone unit. In some embodiments, the battery pack 218 can include a single battery or multiple batteries. In this exemplary embodiment, the battery pack 218 can include a pack of four AA batteries.

In certain embodiments, the microcontroller 216 connects to reset button 220 and to power button 222. The reset button 220 includes an LED indicator ring 224 that can function as a display device to indicate when the surgical safety device 200 needs to be reset. The power button 222 includes an LED indicator ring 226 that can function as a display device to indicate when the surgical safety device 200 has sufficient power or, alternatively, when the surgical safety device 200 is not receiving adequate power.

The reset button 220 can be depressed to reset the surgical safety device 200 after the relay and control board 204 has been triggered to shut down the flow of electricity or information from the cautery console to the surgical instrument. LED indicator ring 224. When triggered, the surgical instrument can be disabled or deactivated to prevent patient injury. The LED indicator ring 224 can emit a red light when the relay and control board 204 has been triggered.

The power button 222 can be depressed to power the surgical safety device 200. Pressing the power button 222 allows power to flow from the battery pack 218 to the microcontroller 216. In this exemplary embodiment, the LED indicator ring 226 emits a white light when the battery pack 218 is powering the microcontroller 216.

With continued reference to the embodiment illustrated in FIGS. 3-5, a microcontroller 216 connects to a pair of EMG boards 228, 230 that are positioned within the housing 202. A first EMG board 228 connects to a first EMG sensor assembly 232 and, alternatively, a first accelerometer assembly 234. A second EMG board 230 connects to a second EMG sensor assembly 236 and a second accelerometer assembly 238. The EMG boards 228, 230 perform essentially the same functions as the EMG boards 128, 130 shown in FIG. 2.

The first EMG sensor assembly 232 includes a plurality of EMG sensor pads 240, 244, cables 246, and a terminal 248 that inserts into the housing 202. The second EMG sensor assembly 236 includes a plurality of EMG sensor pads 250, 254, cables 256, and a terminal 258 that also inserts into the housing 202. The EMG sensor pads 240, 244 and 250, 254 include EMG sensors mounted on pads that can be fixably placed on a patient.

The cables 246, 256 and the terminals 248, 258 connect the sensor assemblies 232, 236 to the EMG boards 228-230. The connection enables the EMG boards 228-230 to communicate with the EMG sensor pads 240-244 and 250-254, so that the EMG boards 228-230 can receive EMG sensor information and process the EMG sensor information to the microcontroller 216. The EMG boards 228-230 can transmit the unprocessed or processed EMG sensor information to the microcontroller 216, so that the microcontroller 216 can detect nerve stimulations and/or muscle movements in patients.

The first accelerometer assembly 234 includes an accelerometer 260, a cable 262, and a terminal 264 that inserts into the housing 202. The second accelerometer assembly 238 also includes an accelerometer 266, a cable 268, and a terminal 270 that inserts into the housing 202.

The cables 262, 268 connect to the accelerometers 260, 266. The terminals 264, 270 connect the cables 262, 268 to the EMG boards 228-230, so that the EMG boards 228, 230 can receive measurements from the accelerometers 260, 266. The EMG boards 228, 230 can process this information with other input from with the EMG sensor pads 240, 244 and 250, 254, which can be transmitted to the microcontroller 216 to facilitate detection of movements or impending movements of the patient's limbs during surgery.

Optionally, the surgical safety device 200 includes a USB connector 272 that is mounted in an opening 274 in the housing 202. The USB connector 272 can perform essentially the same functions as the USB connector 140 shown in FIG. 2. The USB connector 272 can provide power to the microcontroller 216 and/or can transmit information to an external computer system or computing device (not shown).

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain and which fall within the limits of the appended claims.

I claim:

1. A surgical safety device for use in connection with a powered surgical instrument, the surgical safety device comprising:
a controller; the controller in electrical communication with the powered surgical instrument; and
one or a plurality of sensors for detecting unexpected electrical activity in a patient's muscle, the one or a plurality of sensors in electrical communication with the controller;
whereby, upon detection of unexpected electrical activity, the controller is configured to disable the powered surgical instrument.

2. The surgical safety device of claim 1, further comprising one or a plurality of accelerometers for detecting unexpected movement of a patient's limb, wherein the controller is in electrical communication with the one or a plurality of accelerometers.

3. The surgical safety device of claim 2, further comprising a display device for displaying information relating to unexpected movements.

4. The surgical safety device of claim 1, further comprising one or a plurality of electromyography boards in electrical communication with the controller, wherein each board is configured to transmit EMG sensor data that corresponds to the movement of a patient to the controller.

5. The surgical safety device of claim 1, wherein the sensors are electromyography sensors.

6. The surgical safety device of claim 1, wherein the powered surgical instrument is a cautery handpiece.

7. The surgical safety device of claim 1 further comprising a powered tool control console for supplying power to the powered tool, the control console in electrical communication with the controller, whereby the controller can shut off the power to the powered tool if unexpected electrical activity is detected.

8. The surgical safety device of claim 7 further comprising a relay in between the console and the handpiece, wherein the controller is configured to direct the relay to shut off power to the handpiece if unexpected electrical activity is detected.

9. The surgical safety device of claim 1, further comprising a data communication port for exchanging data with the controller.

10. A method for controlling a surgical instrument to prevent injury to a patient, the method comprising the steps of:
sensing unexpected activity in the patient's limb; and
deactivating the surgical instrument.

11. The method of claim 10, wherein the step of sensing unexpected activity is performed with one or more electromyography sensors.

12. The method of claim 11, wherein the one or more electromyography sensors comprise skin surface gelled monitoring electrodes.

13. The method of claim 12, further comprising communicating to a controller that unexpected activity has been detected and deactivating the surgical instrument.

14. The method of claim 10, wherein the step of sensing unexpected activity is performed with one or more accelerometers attached to the patient's limb to detect movement.

15. The method of claim 14, further comprising communicating to a controller that unexpected activity has been detected and deactivating the surgical instrument.

16. The method of claim 10, further comprising displaying output relating to the unexpected activity.

17. A cauterization system comprising:
a cauterizing instrument;
a cautery console for controlling and delivering power to the cauterizing instrument; and
a safety interface for connecting the cauterizing instrument to the cautery console, the safety interface having at least one sensor for monitoring a muscle of interest to detect unexpected activity;
wherein the safety interface deactivates the surgical instrument after receiving an indication from the at least one sensor that indicates that unexpected activity has been detected.

18. The cauterization system of claim 17, wherein the safety interface comprises one or a plurality of accelerometers for detecting unexpected movement.

19. The cauterization system of claim 18, wherein the safety interface comprises one or a plurality electromyography sensors for detecting unexpected electrical activity in a muscle.

20. The cauterization system of claim 17, wherein the safety interface comprises one or a plurality electromyography sensors and one or a plurality of accelerometers.
